(19) United States
(12) Patent Application Publication
(10) Pub. No.: US 2026/0320398 A1
(43) Pub. Date: Oct. 15, 2026

(54) System and Method for Pretraining Octree-Based Transformer Models for Large-Scale 3D Geometry Representation

(71) Applicants: Yalin WANG, Tempe, AZ (US); Mohammad FARAZI, Tempe, AZ (US); Yanxi CHEN, Tempe, AZ (US); Yujian XIONG, Tempe, AZ (US)

(72) Inventors: Yalin WANG, Tempe, AZ (US); Mohammad FARAZI, Tempe, AZ (US); Yanxi CHEN, Tempe, AZ (US); Yujian XIONG, Tempe, AZ (US)

(73) Assignee: Arizona Board of Regents on Behalf of Arizona State University, Scottsdale, AZ (US)

(21) Appl. No.: 19/643,686

(22) Filed: Apr. 9, 2026

Related U.S. Application Data
(60) Provisional application No. 63/786,065, filed on Apr. 9, 2025.

(51) Int. Cl. G06N 3/088; G06N 3/0455; G16H 30/20

(52) U.S. Cl. CPC G06N 3/088; G06N 3/0455; G16H 30/20

(57) ABSTRACT

Pretraining a unified octree transformer pipeline neural network architecture to analyze a plurality of heterogeneous three dimensional (3D) medical tetrahedral and triangular meshes with morphometric features (hereinafter “meshes”) involves receiving the meshes, each comprising a plurality of spatial coordinates, constructing, via octree-guided depth-wise convolution, a plurality of simplex-aware octrees for the meshes, integrating, via a geometry-morphometry fusion module, the morphometric features of the meshes, without architectural changes, and capturing, via a masked autoencoder pretraining pipeline, both geometry features and vertex morphometry of the meshes for any downstream tasks.

### CLAIM OF PRIORITY

This application claims the benefit of U.S. Provisional Patent Application No. 63/786,065, filed Apr. 9, 2025, entitled “SYSTEMS AND METHODS FOR 3D VOLUMETRIC MESH TRANSFORMER”, the disclosure of which is incorporated by reference herein in its entirety.

### COPYRIGHT NOTICE

A portion of this document contains material which is subject to copyright protection. The copyright owner has no objection to the facsimile reproduction by anyone of the document as it appears in the Patent and Trademark Office records but otherwise reserves all copyright rights whatsoever.

### TECHNICAL FIELD

The disclosed embodiments relate to learning from three-dimensional (3D) medical tetrahedral meshes in neuroimaging analysis, in particular, the disclosed embodiments use a unified octree transformer for heterogeneous medical tetrahedral mesh analysis.

### BACKGROUND

Learning from three-dimensional (3D) medical tetrahedral meshes is a fundamental challenge in neuroimaging analysis. Structural Magnetic Resonance Imaging (MRI) has become prominent for its noninvasive nature, high resolution, and suitability for longitudinal studies, playing a critical role in detecting fine-grained deformations such as cortical thinning or volumetric atrophy. Clinical pipelines routinely produce rich per-vertex morphometric signals including cortical thickness, curvature, sulcal depth, and myelin content, which carry critical diagnostic information for conditions such as Alzheimer's disease (AD) and focal cortical dysplasia (FCD).

Traditionally, MRI data are processed as voxel grids which have fixed resolution and are inherently limited in modeling intricate anatomical geometry, while unstructured meshes (tetrahedral/triangular) offer a topologically coherent and expressive alternative for both surface and interior anatomy. Yet existing frameworks are largely restricted to a single mesh type and operate on raw coordinates, ignoring the morphometric features that clinicians rely upon in practice.

In computing, a voxel is a representation of a value on a three-dimensional regular grid, akin to a two-dimensional picture element (pixel). Voxels are frequently used in the visualization and analysis of medical and scientific data (e.g., geographic information systems (GIS)). Volumetric meshes, particularly tetrahedral meshes, provide a more flexible framework for modeling both brain surface and interior structures. In 3D computer graphics and modeling, a volumetric mesh is a polyhedral representation of the interior region of an object. It is unlike polygon meshes, which represent only the surface as polygons. A mesh is a representation of a larger geometric domain by smaller discrete cells. Meshes are commonly used to compute solutions of partial differential equations and render computer graphics. A mesh partitions space into elements (or cells or zones) over which the equations can be solved, which then approximates the solution over the larger domain. Element boundaries may be constrained to lie on internal or external boundaries within a model. Higher-quality (better-shaped) elements have better numerical properties, where what constitutes a “better” element depends on the general governing equations and the solution to the model instance.

Unlike intensity-based representations, tetrahedral meshes provide a structured framework to analyze geometric variations and morphological deformations, enabling a more precise characterization of structural differences across subject groups. A tetrahedral mesh is a representation of a three-dimensional space using tetrahedra. Tetrahedra are polyhedra with four triangular faces, four vertices, and six edges. This type of mesh is commonly used in various fields, including computational fluid dynamics (CFD), finite element analysis (FEA), and computer graphics, to model complex geometries and solve partial differential equations. Morphological deformation refers to the alterations in the shape, size, and structure of an organism, which can occur on a microscopic level within cells or on a macroscopic scale affecting entire organs. These changes are important for understanding how the body functions in both healthy and diseased states. Morphological changes can be caused by various factors, including stress, injury, or genetic mutations. These changes can be visible under a microscope and serve as indicators of cellular health. For example, healthy cells have regular, predictable shape, while damaged or cancerous cells may appear irregular.

### BRIEF DESCRIPTION OF THE DRAWINGS

Embodiments are illustrated by way of example, and not by way of limitation, and can be more fully understood with reference to the following detailed description when considered in connection with the figures in which:

FIGS. 1A and 1B illustrate the disclosed embodiments, referred to herein as OctEncoder, supporting both tetrahedral and triangular meshes, with a flexible choice of representative points that can be adapted to dataset requirements.

FIGS. 2A-2C illustrate details of the three experiments and the auxiliary morphometries used, according to the disclosed embodiments.

FIGS. 3A and 3B illustrate how the disclosed embodiments learn high-quality geometric representations across both mesh domains, with reconstructed points closely matching masked regions even under aggressive masking.

FIG. 4 presents Table 1 which shows AD clinical diagnosis classification, and brain amyloid positivity (Aβ) prediction matched with PET scans and optional pTau-217 measurements.

FIG. 5 presents Table 2 which shows that the disclosed embodiments consistently outperform all baselines across all three pairwise tasks.

FIG. 6 presents Table 3 which shows that the disclosed embodiments substantially outperform the published MELD Graph baseline across all three metrics.

FIG. 7 presents Table 4 which shows each proposed method contributes meaningfully to final performance and confirms the benefit of learning position dependent features adaptively within the octree structure, according to the disclosed embodiments.

### DETAILED DESCRIPTION

Voxel-based and graph-based neural networks generalize 2D CNNs to 3D by discretizing space into regular grids or connectivity graphs, yet both suffer from fundamental scalability limitations. Voxel methods incur cubic memory and computational cost with respect to spatial resolution, a bottleneck that becomes prohibitive for high-resolution medical volumes. Sparse variants mitigate this through octree decompositions or hash-based indexing, with recent architectures further introducing windowed attention over sparse voxels. Graph Neural Networks (GNNs) operate directly on surface mesh topology without spatial discretization, yet predominantly encode neighborhoods as purely topological graphs while neglecting underlying 3D geometry. They are further limited to surface meshes and fail to incorporate per-vertex morphometric features—such as curvature, sulcal depth, or cortical thickness—that carry direct clinical significance in neuroimaging and anatomical shape analysis.

Transformers address these limitations through multi-head self-attention, which enables each geometric primitive—encoded as a token embedding capturing both positional and morphometric attributes—to attend to all others regardless of spatial proximity. Unlike GNNs constrained to fixed local neighborhoods, this global receptive field captures long-range structural dependencies while naturally accommodating irregular, variable-size inputs. These properties make transformers particularly well suited for 3D anatomical shape analysis where local geometry and global morphometric relationships must be jointly modeled.

Point-based Transformers offer strong global modeling but face quadratic complexity on large meshes. Windowed attention reduces cost but assumes regular density, causing information loss under spatially varying mesh structures. OctFormer achieves near-linear complexity via adaptive octree windows yet targets generic point clouds without heterogeneous mesh or morphometric feature support. OctFormer introduced an octree-based self-attention mechanism that partitions point clouds into irregular but equally sized windows, maintaining linear complexity. An octree is a tree data structure in which each internal node has exactly eight children connected to the node via a branch or edge. Octrees are most often used to partition a three-dimensional space by recursively subdividing it into eight octants. OctFormer uses octree-based transformers for 3D point cloud learning. OctFormer provides a backbone for 3D point cloud segmentation and object detection, has linear complexity and is scalable for large-scale point clouds.

Autoencoders emerge as a powerful self-supervised pretraining paradigm, first demonstrated on images by Masked Autoencoders (MAE) and a Simple Framework for Masked Image Modeling (SimMIM). This has been extended to 3D domains: PointMAE—Masked Autoencoders for Point Cloud Self-Supervised Learning—pioneers masked modeling on point clouds with an asymmetric transformer, and PointM2AE introduces multiscale masking for hierarchical geometry learning including a pre-training framework for hierarchical self-supervised learning of 3D point clouds. MAE principles have further been applied to Light Detection and Ranging (LiDAR), Neural Radiance Field (NeRF), an AI technology that transforms 2D images into detailed 3D scenes using deep learning and volumetric rendering, and spatiotemporal data. However, none of these works addresses pretraining on heterogeneous medical meshes with morphometric features, nor exploit shared mesh topology across subjects to amortize structural construction cost: leaving a clear gap for clinical neuroimaging.

A point cloud is a discrete set of unordered 3D points, each represented by Cartesian coordinates (X, Y, Z) and optionally enriched with per-point attributes such as surface normals, RGB color, or intensity values. Point clouds are the native output of 3D acquisition pipelines including LiDAR scanners, structured-light sensors, and photogrammetry systems, and serve as a fundamental representation in applications ranging from autonomous perception and CAD reconstruction to medical image analysis. Unlike voxel grids or meshes, point clouds preserve the raw geometric structure of a scene without imposing discretization or connectivity constraints, making them a flexible but challenging input for deep learning due to their irregular, unordered nature and variable density.

The disclosed embodiments, referred to herein as OctEncoder, use a unified octree transformer pretraining pipeline that addresses these gaps. Contributions of the disclosed embodiments include:
A MAE pretraining pipeline of octree-based geometry for general medical meshes, capturing both geometry features and vertex morphometry for any downstream tasks.
   Multiple simplex-aware octrees construction which supports both tetrahedral and triangular meshes via octree-guided depth-wise convolution.
   A geometry-morphometry fusion module enabling flexible per-vertex clinical feature integration without architectural changes.

### 1. Methods

### 1.1 Simplex-aware Octree Construction

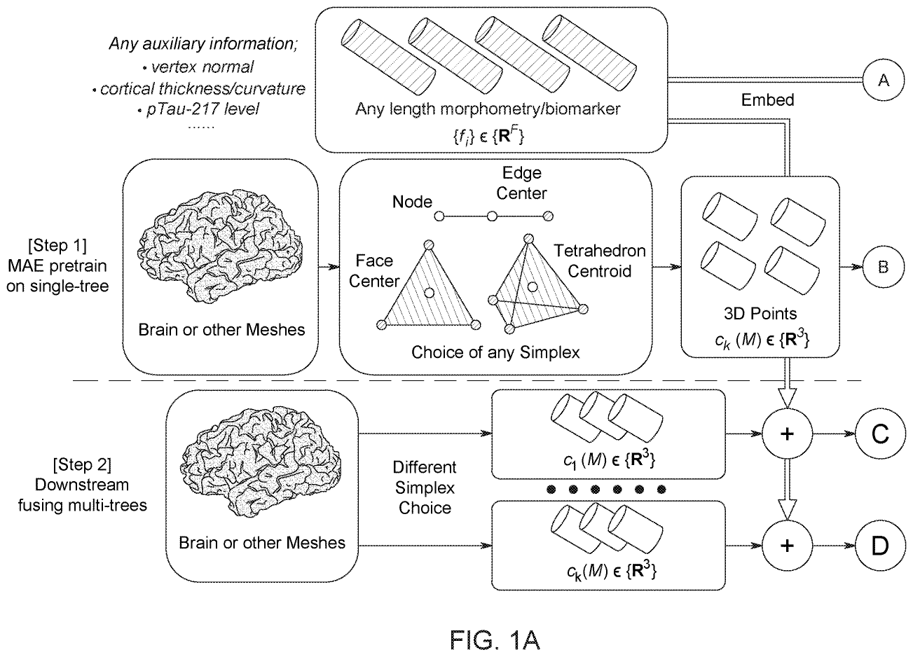

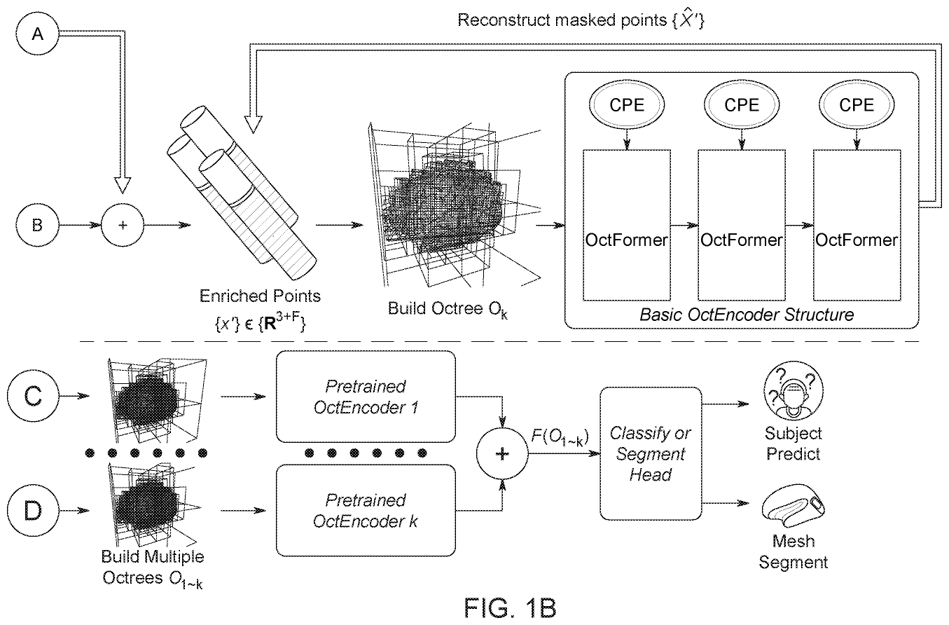

As shown in FIGS. 1A and 1B, OctEncoder supports both tetrahedral and triangular meshes, with a flexible choice of representative points that can be adapted to dataset requirements. Formally, given a mesh M=(V, S) where V is the vertex set and S the set of simplices, a representative point function c: M→{R³} is defined that produces a set of spatially localized points from the mesh.

For complex meshes, the pipeline can construct multiple complementary octrees. For example, the first uses tetrahedron centroids to capture volumetric interior geometry, while the second uses mesh vertices directly:

$${c}_{1} ( ℳ ) = \{ \frac{1}{4} {\sum }_{{v}_{i} ∈ t} {v}_{i} , t ∈ 𝒮 \} , {c}_{2} ( ℳ ) = \{ {v}_{i} , {v}_{i} ∈ 𝒱 \} , … , {c}_{K} ( ℳ )$$

The octree Oₖ is constructed by inserting all representative points x∈cₖ(M) up to a user-defined depth d, and nodes are ordered along a 3D Z-order space-filling curve for memory-contiguous window partitioning and efficient parallel construction. More generally, octrees can be constructed from any choice of simplex (vertex, edges/faces/tetrahedron center, etc.), and a learned weighted linear fusion strategy F(O₁, . . . , O_{K}) merges outputs across branches. The specific choice of simplex and number of octrees is a design decision driven by the dataset and task; the configuration reflects the setting used in the experiments described herein.

Conditional Positional Encoding (CPE) is applied independently within each octree branch before fusion, allowing each branch to develop spatially aware embeddings prior to interaction.

### 1.2 Auxiliary Clinical Feature Embedding

Beyond constructing octrees from spatial coordinates alone, each vertex vᵢ∈V may carry a set of F auxiliary feature channels derived from neuroimaging or geometry-morphometry pipelines, such as cortical thickness, curvature, etc. Given a per-vertex feature vector fᵢ∈R^{F}, any combination of per-vertex attributes can be incorporated, and the number of channels F is flexible.

To embed such morphometric information into the octree representation, each representative point x∈cₖ(M) is augmented by projecting the concatenation of its spatial coordinate and feature vector through a learnable linear layer:

$${x}^{′} = W [ \begin{matrix}x \\ {f}_{i}\end{matrix} ] + b$$

where x′∈R^{3+F} is the resulting enriched embedding. This allows OctEncoder to accommodate varying neuroimaging features without architectural changes.

### 1.3 Octree Attention, MAE Pretraining and Downstream

Transformer attention is computed within local windows defined by the octree's Z-order partitioning, keeping attention complexity near-linear in the number of nodes. Dilated attention complements local windows by sampling tokens at a fixed stride across the octree, enlarging the receptive field without additional memory cost. Each transformer block follows a standard residual design with layer normalization and a feedforward MLP.

For pretraining on large unlabeled brain surfaces, a masked autoencoder strategy is adopted that is tailored to octree-structured data. A fixed proportion of octree tokens are masked, and the encoder processes only the visible subset to produce latent representations. A lightweight transformer decoder then reconstructs the masked tokens \hat{x}′=[\hat{x}, \hat{f}], supervised by a hybrid loss combining both Chamfer Distance over coordinates and MSE loss over morphometric features:

$$ℒ = {\underbrace{{\sum }_{p ∈ ( \hat{x} )} {min}_{q ∈ ( x )} {‖ p - q ‖}^{2} + {\sum }_{q ∈ \{ x \}} {min}_{p ∈ ( \hat{x} )} {‖ p - q ‖}^{2}}}_{{ℒ}_{chamfer}} + λ \underbrace{\frac{1}{\vert ℳ \vert} {\sum }_{i ∈ ℳ} {‖ {\hat{f}}_{i} - {f}_{i} ‖}^{2}} \text{?}$$

$$\text{?} \text{indicates text missing or illegible when filed}$$

After pretraining, the encoder serves as a general backbone that can be coupled with any task-specific head for downstream applications such as AD diagnosis or FCD segmentation. This flexibility allows OctEncoder to serve as a unified backbone for clinical neuroimaging tasks without retraining from scratch.

### 2. Experimental Design

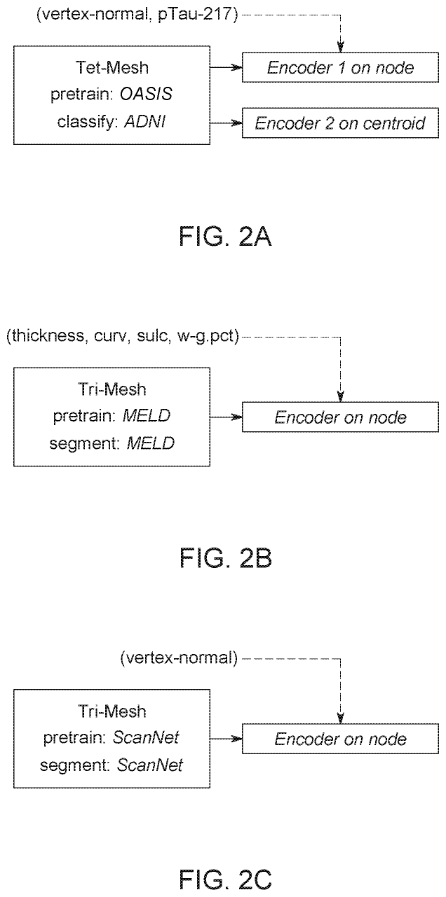

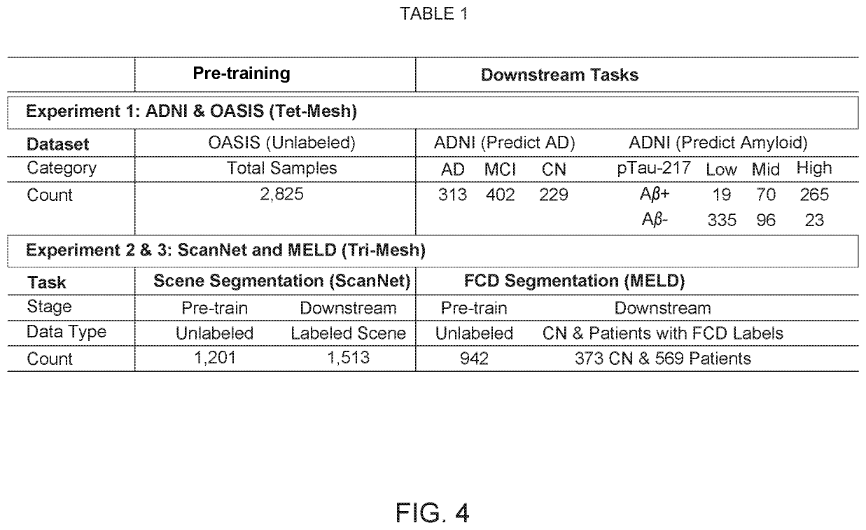

ADNI Classification: the MAE encoder is first trained on the OASIS3 dataset, which provides largescale unlabeled sMRI scans across multiple sessions and subjects. The encoder is then used on the Alzheimer's Disease Neuroimaging Initiative (ADNI) dataset for two downstream tasks, as summarized in Table 1 presented in FIG. 4: AD clinical diagnosis classification, and brain amyloid positivity (Aβ) prediction matched with PET scans and optional pTau-217 measurements. All MRIs are processed using FreeSurfer to reconstruct cortical surfaces, and volumetric tetrahedral meshes are generated via TetGen between pial and white-matter surfaces, producing approximately 130k-150k vertices per subject. Ground truth Aβ are derived from PET Centiloid values, where subjects with Centiloid>20 are classified as Aβ positive. The pTau-217 biomarker, quantified via the PrecivityAD2 assay, serves as an auxiliary biochemical label of Aβ prediction. Details of the three experiments and the auxiliary morphometries used are provided in FIGS. 2a-2c. AD classification conducts pairwise binary tasks among AD/MCI/CN groups, comparing against tetrahedral mesh baselines including ChebyNet, GAT, and TetCNN. For amyloid prediction, the disclosed embodiments evaluate on mesh only inputs and with auxiliary pTau-217 labels, comparing against logistic regression baselines using hippocampal volume and pTau-217. All models are evaluated using accuracy, sensitivity, and specificity.

MELD Segmentation: FCD is a leading cause of drug-resistant focal epilepsy, yet lesions are frequently MRI negative and challenging to delineate precisely. OctEncoder was evaluated on the publicly available MELD dataset, a large multicenter cohort with cortical surface meshes processed via FreeSurfer to extract 34 per-vertex surface-based features including cortical thickness, gray-white matter intensity contrast, intrinsic curvature, sulcal depth, and FLAIR intensity sampled at multiple cortical depths. Each hemisphere is represented as a triangular surface mesh with 164k vertices and a corresponding binary lesion mask as the segmentation target. The published MELD Graph results are adopted as the primary baseline for the disclosed embodiments. Performance is evaluated using subject-level sensitivity, specificity, and vertex-level lesion Intersection over Union (IoU).

ScanNet Segmentation: To test generalizability of the disclosed embodiments beyond medical domains, OctEncoder was evaluated on largescale 3D semantic segmentation using ScanNet, comprising reconstructed indoor scenes annotated with 20 semantic categories. Only face normals were used as auxiliary morphometries. Following standard protocol, training scenes were used for MAE pretraining and finetuning, with held out scenes for validation and testing. The disclosed embodiments were compared against OctFormer, Point Transformer V1-V3, Mix3D, OCNN, and TTTKD, evaluated using mean Intersection-over-Union (mIoU).

### 3. Results

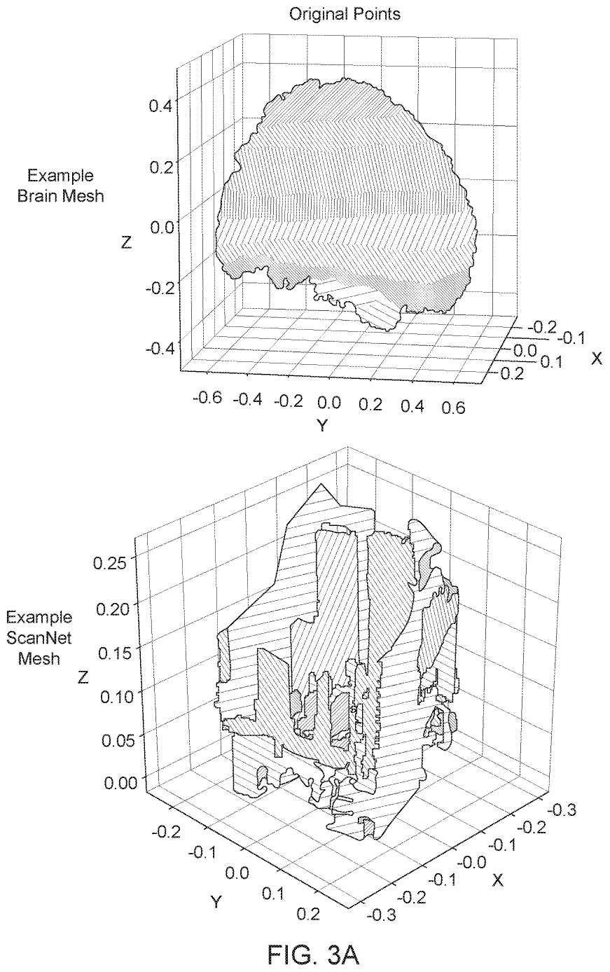

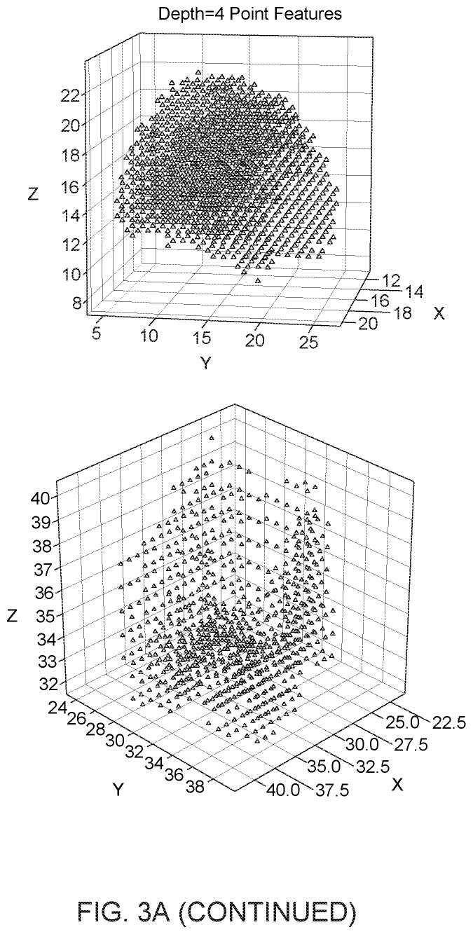

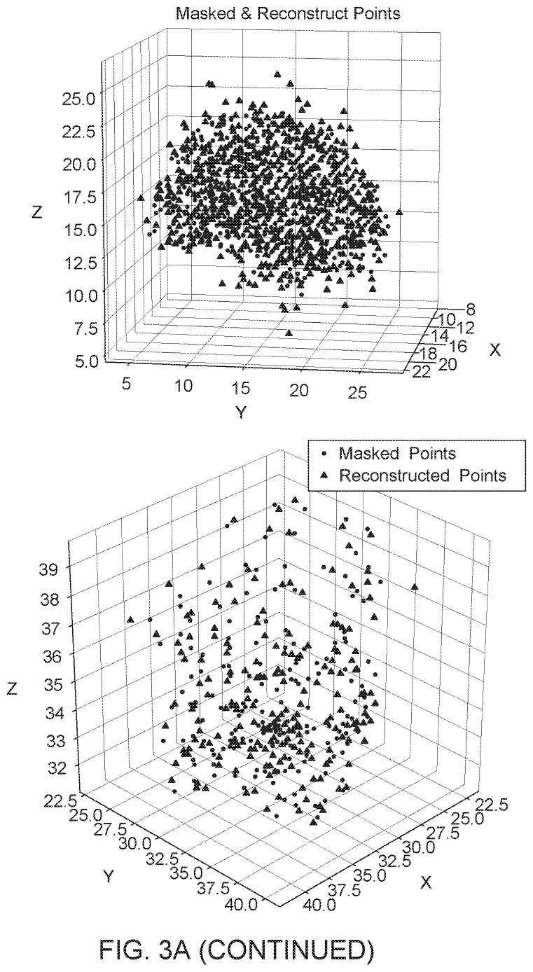

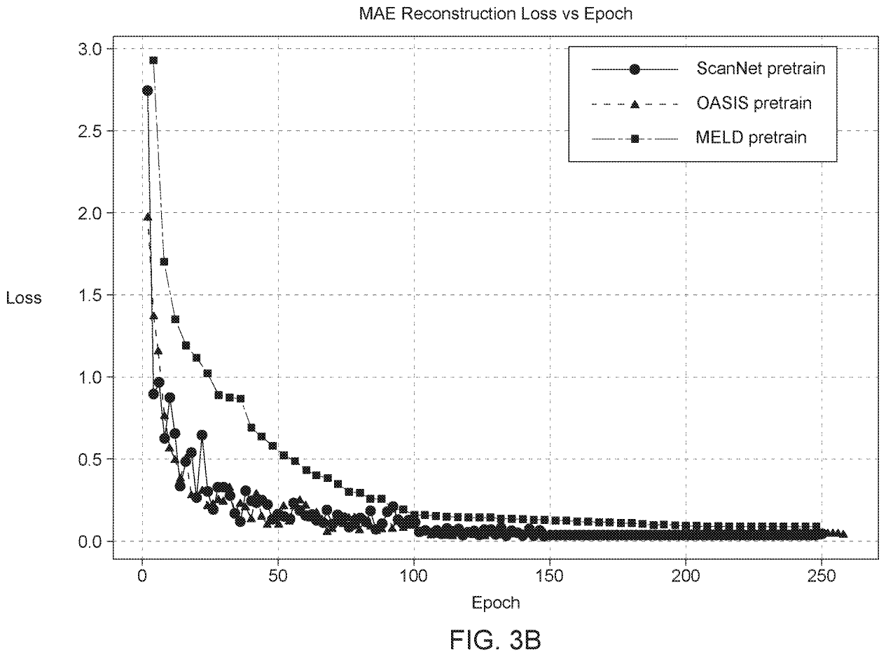

MAE Pretraining Performance. As shown in FIGS. 3A and 3B, OctEncoder learns high-quality geometric representations across both mesh domains, with reconstructed points closely matching masked regions even under aggressive masking. Both pretraining curves converge steadily within roughly 100 epochs, after which additional epochs contribute negligible reconstruction improvement. FIG. 3A illustrates visualization of MAE pretraining on brain tet-meshes (top) and ScanNet tri-meshes (bottom). From left: Original Points, octree Depth=4-point features, and masked input (circular dots) with reconstruction (triangles). FIG. 3B illustrates MAE reconstruction loss curves for OASIS pretraining, ScanNet pretraining, and MELD pretraining.

Computation Costs. All brain experiments were run on a single NVIDIA Quadro RTX 5000 GPU, where MAE pretraining took approximately 1.5 GPU hours and downstream finetuning completed in around 2 hours. ScanNet experiments were run on 4 identical GPUs, with MAE pretraining requiring approximately 20 GPU hours and segmentation training approximately 33 GPU hours.

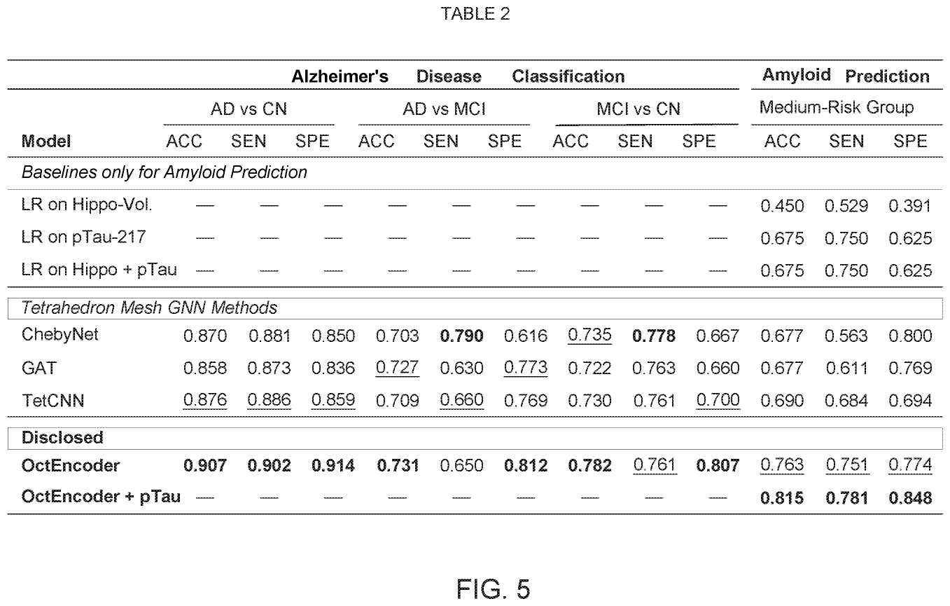

AD Classification. OctEncoder consistently outperforms all baselines across all three pairwise tasks (see Table 2 presented in FIG. 5). For AD vs. CN, the model achieves strong accuracy and well-balanced sensitivity and specificity, confirming robust discrimination between late-stage Alzheimer's and healthy controls. The most clinically significant result is in MCI vs. CN, where OctEncoder improves accuracy by 4.7% over the second-best model, demonstrating superior sensitivity to early and subtle pathological differences in prodromal subjects: a task of direct relevance for identifying individuals at risk of progression to AD. In AD vs. MCI, ChebyNet achieves slightly higher sensitivity, but at the cost of substantially lower specificity, reflecting a tendency to overpredict positives rather than reliably discriminate between groups.

Amyloid Positivity Prediction. The focus is on the medium risk subgroup, where amyloid status is the most ambiguous and biomarker-based prediction is the most clinically uncertain. Notably, adding hippocampal volume to pTau-217 in logistic regression yields no improvement, suggesting these two conventional features are largely redundant in this subgroup. OctEncoder alone surpasses all biomarker-only baselines with balanced sensitivity and specificity, demonstrating that tetrahedral mesh geometry captures pathological signals not reflected in scalar biomarkers. Its fusion with pTau-217 achieves the strongest overall performance, with accuracy of 0.815, confirming that structural mesh features and blood-based biomarkers carry complementary information for identifying amyloid pathology in clinically ambiguous cases.

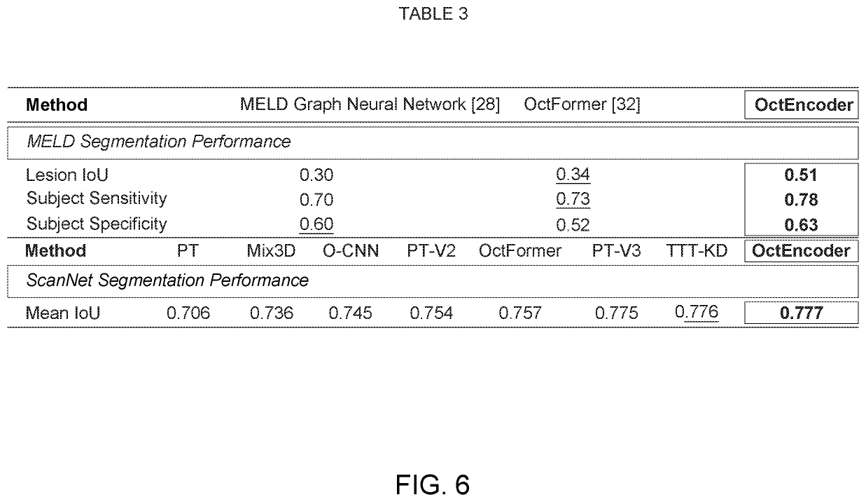

FCD Lesion Segmentation. OctEncoder substantially outperforms the published MELD Graph baseline across all three metrics (see Table 3 presented in FIG. 6). The most striking improvement is in a lesion IoU, which increases from 0.30 to 0.51, reflecting significantly more precise delineation of dysplastic cortical regions. Subject-level sensitivity also improves from 0.70 to 0.78, indicating that OctEncoder detects a greater proportion of patients with FCD. Specificity improves more modestly, suggesting the primary gain is in lesion localization accuracy rather than false positive reduction. These results demonstrate that the octree transformer architecture, equipped with rich per-vertex morphometric features and MAE pretraining on unlabeled cortical meshes, is well suited to the subtle and spatially irregular patterns characteristic of FCD.

3D Semantic Segmentation. OctEncoder achieves the highest mIoU among all compared methods on ScanNet, marginally surpassing TTTKD and Point Transformer V3 while offering substantially reduced computational cost through octree-guided encoding and MAE pretraining. The consistent improvement over earlier transformer-based models including OctFormer and Point Transformer V1/V2 confirms the benefit of combining hierarchical octree partitioning with masked pretraining for dense semantic segmentation in largescale indoor scenes.

### 3.1 Ablation Studies

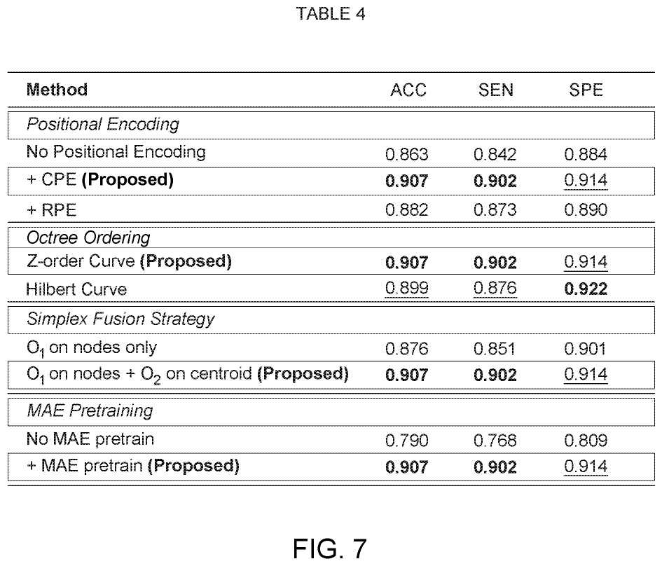

Due to page constraints, ablation studies are reported on the AD vs. CN task only. As Table 4, presented in FIG. 7, graphically illustrates, each proposed component contributes meaningfully to final performance. CPE yields the largest positional encoding gain over both the no encoding baseline and conventional RPE, confirming the benefit of learning position dependent features adaptively within the octree structure. For octree ordering, Z-order and Hilbert curves perform comparably, but Z-order is preferred for its better computational efficiency at scale. Fusing node and tetrahedron center octrees (O1, O2) improves over the single octree variant, demonstrating the value of multi-view geometric aggregation. Removing MAE pretraining causes the largest single performance drop across all ablations, confirming that self-supervised pretraining on large unlabeled meshes is foundational rather than merely supplementary for high quality mesh representation learning.

### 4. Conclusion

The disclosed embodiments, referred to herein as OctEncoder, is a unified octree transformer for heterogeneous medical mesh analysis. Beyond OctFormer, the disclosed embodiments provide the following advances: simplex-aware multitree construction supporting both tetrahedral and triangular meshes via flexible simplicial complex fusion; a geometry-morphometry embedding that integrates arbitrary per-vertex clinical features without architectural changes; and a general MAE pretraining pipeline that jointly reconstructs geometry and morphometry across any mesh modality, exploiting shared topology to reduce pretraining cost. Validated across three clinically distinct tasks on ADNI tetrahedral brain meshes, MELD cortical surface meshes, and ScanNet indoor scenes, OctEncoder achieves state of the art performance in all settings, confirming that unified geometry-morphometry encoding with self-supervised pretraining is both clinically effective and broadly generalizable.

In some embodiments, a deep learning model can be constructed or otherwise trained based on training data using one or more different learning techniques, such as supervised learning, unsupervised learning, reinforcement learning, ensemble learning, active learning, transfer learning, or other suitable learning techniques for deep learning models. As an example, supervised learning involves presenting a computer system with example inputs and their actual outputs (e.g., categorizations). In some embodiments, the deep learning model is configured to learn a general rule or model that maps the inputs to the outputs based on the provided example input-output pairs.

In some embodiments, the one or more trained deep learning models are then stored for later use. In some embodiments, storing the deep learning model(s) may include storing parameters (e.g., weights, biases, or both), which have been computed or otherwise estimated by training the deep learning model(s) on the training data. In some embodiments, storing the trained deep learning model(s) may also include storing the deep learning model architecture to be implemented. In some embodiments, data pertaining to layers in a neural network architecture (e.g., number of layers, type of layers, ordering of layers, connections between layers, hyperparameters for layers) may be stored.

The disclosed embodiments contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. The processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the disclosure provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk, read-only memories (ROMs), random access memories (RAMs), EPROMs, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. On the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the claims that follow, along with the full scope of equivalents to which such claims are entitled.

What is claimed is:

1. A method for pretraining a unified octree transformer pipeline neural network architecture to analyze a plurality of heterogeneous three dimensional (3D) medical tetrahedral and triangular meshes with morphometric features (hereinafter “meshes”), comprising:
receiving the meshes, each comprising a plurality of spatial coordinates;
constructing, via octree-guided depth-wise convolution, a plurality of simplex-aware octrees for the meshes;
integrating, via a geometry-morphometry fusion module, the morphometric features of the meshes, without architectural changes; and
capturing, via a masked autoencoder pretraining pipeline, both geometry features and vertex morphometry of the meshes for any downstream tasks.

2. The method of claim 1, further comprising defining a representative point function that produces a set of spatially localized points from one of the meshes.

3. The method of claim 2, wherein given a mesh M=(V, S) where V is a vertex set and S is a set of simplices, a representative point function c:M→{R³} is defined that produces the set of spatially localized points from the one of the meshes.

4. The method of claim 1 wherein constructing, via octree-guided depth-wise convolution, the plurality of simplex-aware octrees for the meshes comprises constructing multiple complementary octrees, including a first octree that uses tetrahedron centroids to capture volumetric interior geometry, and second octree that directly uses mesh vertices.

5. The method of claim 1, wherein constructing, via octree-guided depth-wise convolution, the plurality of simplex-aware octrees for the meshes comprises constructing octrees from one of a vertex, an edge, a face, and a tetrahedron center simplex.

6. The method of claim 5, wherein constructing the plurality of simplex-aware octrees comprises constructing for each octree a tree data structure in which each internal node has exactly eight children connected to the node via a respective branch, the method further comprising merging a learned weighted linear fusion strategy across branches of the octrees.

7. The method of claim 6, further comprising independently applying conditional positional encoding within each octree branch before merging the learned weighted linear fusion strategy across branches of the octrees, allowing each branch to develop spatially aware embeddings.

8. The method of claim 1, further comprising adopting a masked autoencoder strategy for pretraining on large unlabeled brain surfaces.

9. The method of claim 8, wherein adopting the masked autoencoder strategy for pretraining on large unlabeled brain surfaces comprises masking a fixed proportion of octree nodes and processing only a visible subset of the octree nodes to produce latent representations.

10. The method of claim 9, further comprising reconstructing the masked octree nodes, via a lightweight transformer decoder.

11. The method of claim 10, further comprising supervising, via a hybrid loss, combining both Chamfer Distance over the spatial coordinates of the meshes and a Mean Square Error (MSE) loss over morphometric features of the meshes.

12. The method of claim 1, further comprising, after pretraining the unified octree transformer pipeline neural network architecture, coupling the unified octree transformer pipeline neural network architecture as a general backbone with a task-specific head for downstream applications.

13. The method of claim 12, wherein coupling the unified octree transformer pipeline neural network architecture as the general backbone with the task-specific head for downstream applications comprises coupling the unified octree transformer pipeline neural network architecture as the general backbone with the task-specific head for clinical neuroimaging tasks without retraining the unified octree transformer pipeline neural network architecture from scratch.

14. The method of claim 13, wherein coupling the unified octree transformer pipeline neural network architecture as the general backbone with the task-specific head for clinical neuroimaging tasks without retraining the unified octree transformer pipeline neural network architecture from scratch comprises coupling the unified octree transformer pipeline neural network architecture as the general backbone with the task-specific head for clinical diagnostic tasks associated with Alzheimer's disease (AD) and focal cortical dysplasia (FCD) without retraining the unified octree transformer pipeline neural network architecture from scratch.

15. A system comprising:
a memory to store instructions;
a processor to execute the instructions stored in the memory to pretrain a unified octree transformer pipeline neural network architecture to analyze a plurality of heterogeneous three dimensional (3D) medical tetrahedral and triangular meshes with morphometric features (hereinafter “meshes”), comprising:
receiving the meshes, each comprising a plurality of spatial coordinates;
constructing, via octree-guided depth-wise convolution, a plurality of simplex-aware octrees for the meshes;
integrating, via a geometry-morphometry fusion module, the morphometric features of the meshes, without architectural changes; and
capturing, via a masked autoencoder pretraining pipeline, both geometry features and vertex morphometry of the meshes for any downstream tasks.

16. The system of claim 15 wherein constructing, via octree-guided depth-wise convolution, the plurality of simplex-aware octrees for the meshes comprises constructing multiple complementary octrees, including a first octree that uses tetrahedron centroids to capture volumetric interior geometry, and second octree that directly uses mesh vertices.

17. The system of claim 15, wherein constructing, via octree-guided depth-wise convolution, the plurality of simplex-aware octrees for the meshes comprises constructing octrees from one of a vertex, an edge, a face, and a tetrahedron center simplex.

18. A non-transitory computer readable storage media having instructions stored thereupon that, when executed by a system having at least a processor and a memory therein, cause the processor to pretrain a unified octree transformer pipeline neural network architecture to analyze a plurality of heterogeneous three dimensional (3D) medical tetrahedral and triangular meshes with morphometric features (hereinafter “meshes”), comprising:
receiving the meshes, each comprising a plurality of spatial coordinates;
constructing, via octree-guided depth-wise convolution, a plurality of simplex-aware octrees for the meshes;
integrating, via a geometry-morphometry fusion module, the morphometric features of the meshes, without architectural changes; and
capturing, via a masked autoencoder pretraining pipeline, both geometry features and vertex morphometry of the meshes for any downstream tasks.

19. The non-transitory computer readable storage media of claim 18 wherein constructing, via octree-guided depth-wise convolution, the plurality of simplex-aware octrees for the meshes comprises constructing multiple complementary octrees, including a first octree that uses tetrahedron centroids to capture volumetric interior geometry, and second octree that directly uses mesh vertices.

20. The non-transitory computer readable storage media of claim 18, wherein constructing, via octree-guided depth-wise convolution, the plurality of simplex-aware octrees for the meshes comprises constructing octrees from one of a vertex, an edge, a face, and a tetrahedron center simplex.
